(19) United States
(12) Patent Application Publication
(10) Pub. No.: US 2016/0243329 A1
(43) Pub. Date: Aug. 25, 2016

(54) HIGH FLOW VENTILATION SYSTEM FOR ENDOSCOPY PROCEDURES

(71) Applicant: Alexander C. Chen, Saint Louis, MO (US)

(72) Inventor: Alexander C. Chen, Saint Louis, MO (US)

(21) Appl. No.: 15/047,491

(22) Filed: Feb. 18, 2016

Related U.S. Application Data
(60) Provisional application No. 62/117,852, filed on Feb. 18, 2015.

(51) Int. Cl. A61M 16/08; A61B 1/273; A61B 1/24; A61B 1/267; A61M 16/12; A61M 16/00

(52) U.S. Cl. CPC A61M 16/0833; A61M 16/12; A61M 16/0096; A61M 16/0057; A61B 1/24; A61B 1/2676; A61B 1/2736; A61M 2202/0208

(57) ABSTRACT

A high flow ventilation system delivers oxygen-enriched air to a patient at flow rates greater than 10 L/min using only conventional wall flow meters as found in most hospitals. The ventilation system combines the output of a plurality of wall flow meters into a specially configured gas mixing apparatus. The gas mixing apparatus provides oxygen-enriched air with an FᵢO₂ (fraction of inspired oxygen) of approximately 21-100% with a total oxygen flow rate of 10-20 L/min or higher as required for high flow ventilation therapy. The oxygen-enriched air is delivered to the patient through a bite block device that allows insertion of an endoscope while simultaneously delivering high flow ventilation therapy to the patient through a large annular air flow passage around the endoscope.

### CROSS REFERENCE TO OTHER APPLICATIONS

This application claims priority of U.S. Provisional Application 62/117,852, filed on Feb. 18, 2015. This and all patents and patent applications referred to herein are incorporated by reference in their entirety.

### FIELD OF THE INVENTION

The present invention relates generally to ventilation systems for delivering breathing gases to a patient. More particularly, it relates to a high flow ventilation system for delivering oxygen-enriched air to a patient during endoscopy procedures, such as bronchoscopy or gastrointestinal endoscopy.

### BACKGROUND OF THE INVENTION

Medical procedures are often performed using some form of sedation, ranging from mild anxiolysis to general anesthesia. While under the effects of sedation, a patient's respiratory drive may diminish, which can lead to lower levels of circulating oxygen. This diminished respiratory drive resulting in lower circulating oxygen is often managed by providing patients with supplemental oxygen during the procedure, often through a facemask or through a nasal cannula. Oxygen flow is controlled using a wall-mounted flow meter connected to a central pressurized oxygen supply system in the hospital, which allows the medical team to titrate oxygen flow going to the patient. Conventional flow meters found in most hospitals measure oxygen flow from 0-10, or sometimes 0-15, liters per minute (L/min), providing a measured flow of supplemental oxygen to patients during medical procedures. While this flow rate of oxygen may be adequate to support patients in some cases, it may not be adequate to support patients in instances where medical procedures are more complicated or prolonged, placing patients at risk for potential complications such as cardiovascular or respiratory compromise.

Higher oxygen flow rates than those provided by the conventional wall-mounted flow meter will generally require a higher pressure oxygen source and a specialized high flow oxygen mixing and delivery system. These requirements can add significantly to the cost of ventilation therapy. Furthermore, while most hospital rooms are equipped with more than one standard wall oxygen flow meter, most are not equipped with high pressure oxygen sources.

Endoscopy procedures, such as bronchoscopy or gastrointestinal endoscopy, may further compromise the ability of the patient to maintain adequate oxygenation because the endoscope partially occludes the airway during the procedure. Thus supplemental oxygen is of particular importance in these procedures.

In instances where oxygen flow is insufficient to support patients, more invasive means of respiratory support may be required, such as endotracheal intubation and mechanical ventilation. These more invasive ventilation methods incur additional cost and add incrementally to the complexity and risk of the medical procedure. Furthermore, the presence of an endotracheal tube will interfere with the ability to perform endoscopy on the patient.

Accordingly, a device which facilitates increased oxygen delivery to patients without requiring endotracheal intubation would be desirable for patients undergoing medical procedures, particularly those involving bronchoscopy or gastrointestinal endoscopy. It is preferable that this be accomplished with the use of conventional wall flow meters and without costly apparatus.

The high flow ventilation system described herein may also be used for patients in acute respiratory distress as a temporizing measure while arrangements for invasive (endotracheal intubation) or non-invasive ventilation (continuous positive airway pressure) are made.

### SUMMARY OF THE INVENTION

In keeping with the foregoing discussion, the present invention provides a high flow ventilation system for delivering oxygen-enriched air to a patient that provides oxygen flow rates greater than 10 L/min using only conventional wall flow meters as found in most hospitals. This is accomplished by combining the output of a plurality of wall flow meters into a specially configured gas mixing apparatus. The gas mixing apparatus provides oxygen-enriched air with an FᵢO₂ (fraction of inspired oxygen) of approximately 21-100% with a total oxygen flow rate of 10-20 L/min or higher as required for high flow ventilation therapy.

In one preferred embodiment, the oxygen-enriched air from the gas mixing apparatus is delivered to the patient through a specially configured bite block device that allows insertion of an endoscope while simultaneously delivering high flow ventilation therapy to the patient. A bite block is an apparatus designed to be placed between the upper and lower jaw to keep the patient's mouth open during medical procedures, such as bronchoscopy or gastrointestinal endoscopy. The configuration of the bite block device creates a large annular air flow passage around the endoscope, which facilitates the delivery of oxygen-enriched air to the lungs. Conventional bite blocks designed for endoscopy do not provide an air passage for ventilating the patient, requiring that supplemental oxygen be delivered to the patient through a nasal cannula or other means.

Alternatively, the gas mixing apparatus of the invention can also deliver high-flow, oxygen-enriched air to a patient using a laryngeal mask airway, an endotracheal tube, an endoscopy mask or a standard ventilation mask.

### BRIEF DESCRIPTION OF THE DRAWINGS

FIG. 1 is a schematic drawing of a gas mixing apparatus in accordance with the invention.

FIGS. 2A-2E are schematic drawings of the components of a bite block device in accordance with the invention.

FIG. 3 is a schematic drawing of the bite block device assembled.

FIG. 4 is a schematic drawing of the gas mixing apparatus and the bite block device assembled together.

FIGS. 5A-5D are drawings of a preferred embodiment of the gas mixing apparatus. FIG. 5A is a front view, FIG. 5B is a side view, FIG. 5C is a first end view and FIG. 5D is a second end view of the gas mixing apparatus 100.

FIGS. 6A-6F are drawings of the components of a preferred embodiment of the bite block device.

FIG. 7A is a perspective view showing a version of a T-piece adapter with a bite block insert integrated directly into it.

FIG. 7B is a phantom view showing the interior structure of the T-piece adapter of FIG. 7A.

FIG. 7C is a cutaway view showing a cross section of the T-piece adapter of FIG. 7A.

### DESCRIPTION OF THE INVENTION

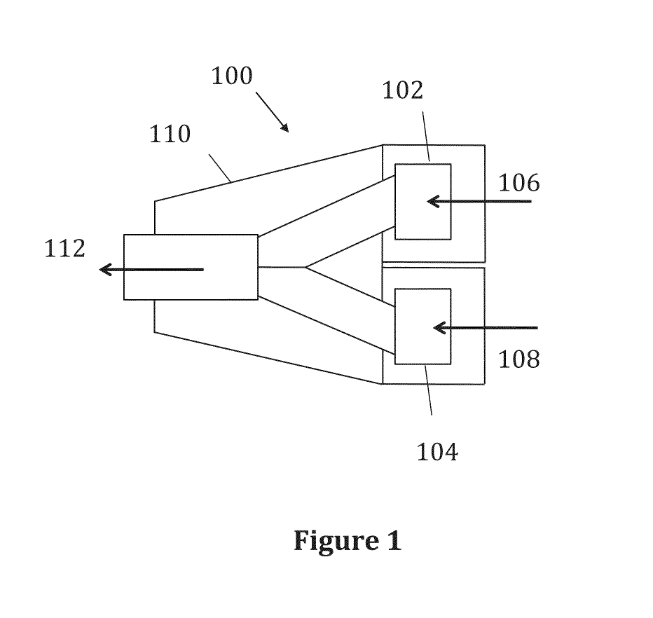

FIG. 1 is a schematic drawing of a gas mixing apparatus 100 in accordance with the invention. The gas mixing apparatus 100 utilizes a plurality of jet mixing devices 102, 104 for mixing pure oxygen with ambient air at the desired ratio. Each jet mixing device has a tubing barb 106, 108 that allows it to be connected via tubing to a standard wall oxygen flow meter. A jet mixing device, also commonly known as a Venturi device, is a gas mixing device that directs a flow of pressurized oxygen through a small diameter jet orifice positioned in a gas mixing chamber within a tubular housing with one or more air entrainment ports in the wall of the tubular housing. When the oxygen exits the jet orifice, ambient air is entrained through the ports into the housing and mixes with the oxygen in the gas mixing chamber. The size of the air entrainment ports regulates the ratio of oxygen to ambient air. Jet mixing devices or Venturi devices that can be used in the gas mixing apparatus are commercially available having fixed ratios from 24% to 60% oxygen or with an adjustable ratio from 24% to 60% oxygen. Commercially available jet mixing devices or Venturi devices are generally limited in the flow rate of oxygen that they are capable of delivering. The output from a plurality of jet mixing devices is therefore combined to provide oxygen flow rates greater than 10 L/min.

A range of oxygen flow rate from 10 to 20 L/min is adequate for most applications. Thus, in the example shown in FIG. 1, the gas mixing apparatus 100 has a first jet mixing device 102 and a second jet mixing device 104 that are connected to a Y-connector 110, which acts as a manifold to combine the output from the two jet mixing devices. If higher oxygen flow rates are desired, three or more jet mixing devices can be combined with an appropriately shaped manifold. A large diameter flexible tube is used to connect the outflow port 112 of the Y-connector 110 to the bite block device to direct the oxygen-enriched air from the gas mixing apparatus to the patient.

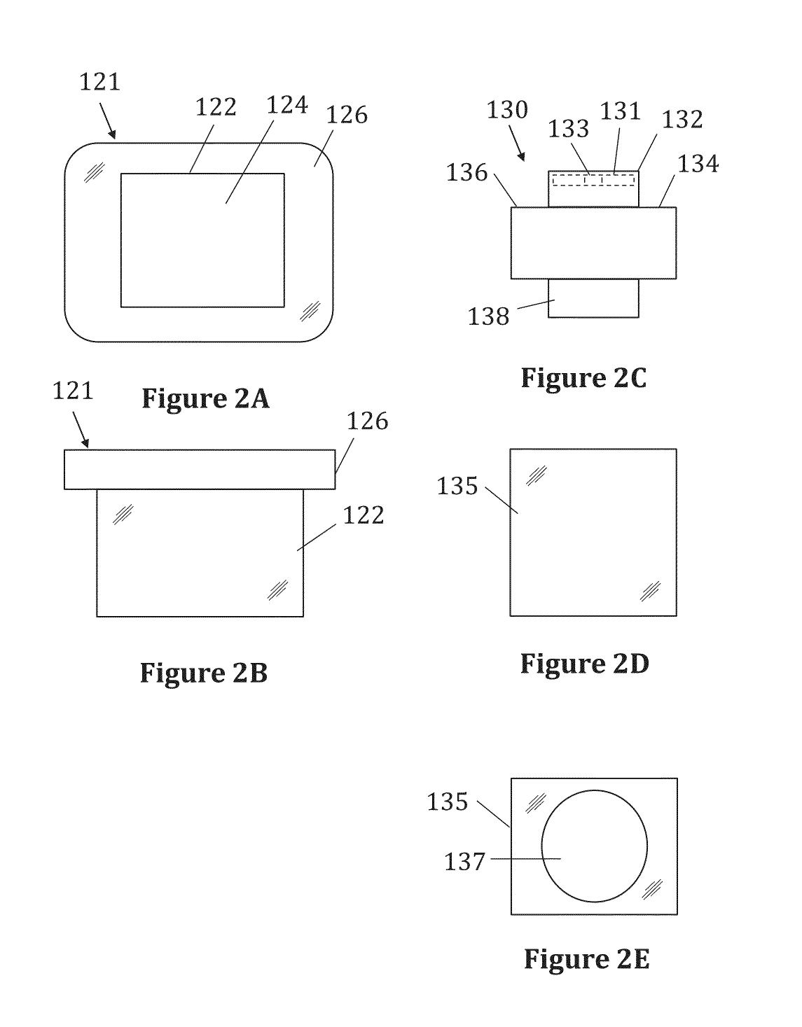

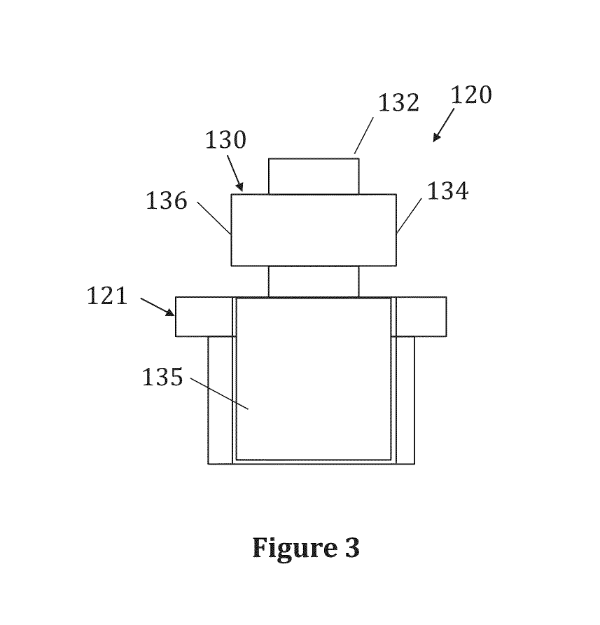

FIGS. 2A-2E are schematic drawings of the components of a bite block device 120 in accordance with the invention and FIG. 3 is a schematic drawing of the bite block device 120 assembled. The bite block device 120 includes a bite block component 121, a T-piece adapter 130 and a bite block insert 135. FIG. 2A shows a top view and FIG. 2B shows a side profile view of the bite block component 121. The bite block component 121 has a tubular body 122 with a flange 126 on one end of the tubular body. As seen in the top view of FIG. 2A, there is a central passage 124 through the tubular body 122 of the bite block component 121. The central passage 124 and the tubular body 122 can have an approximately rectangular cross section as shown, or alternatively they may each have a round, oval, oblong or other desired cross-sectional shape. FIG. 2D shows a top view and FIG. 2E shows a side profile view of the bite block insert 135 that facilitates connection of the T-piece adapter 130, shown in FIG. 2C, to the bite block component 121. In the example shown, the bite block insert 135 has a rectangular external shape that is adapted to insert into the central passage 124 of the bite block component 121 and a circular internal passage 137 that is shaped for connection to a connection port 138 of the T-piece adapter 130, which is shown in FIG. 2C. Alternatively, the T-piece adapter 130 can be configured to connect directly to the bite block component 121 or these two components can be integrated together.

At the top of the T-piece adapter 130 is an endoscope port 132 that is sized and shaped for insertion of an endoscope. The endoscope port 132 has a flexible diaphragm 131 with a central opening 133 or other sliding seal to seal around the body of the endoscope when it is inserted, while allowing longitudinal movement of the endoscope. On the right hand side of the T-piece adapter 130 is an inlet port 134 that is adapted for connection to the outflow 112 of the gas mixing apparatus 100 via a piece of flexible tubing. On the left hand side of the T-piece adapter 130 is an outlet port 136 that is adapted for connection to an air reservoir, for example an expandable/collapsible breathing bag.

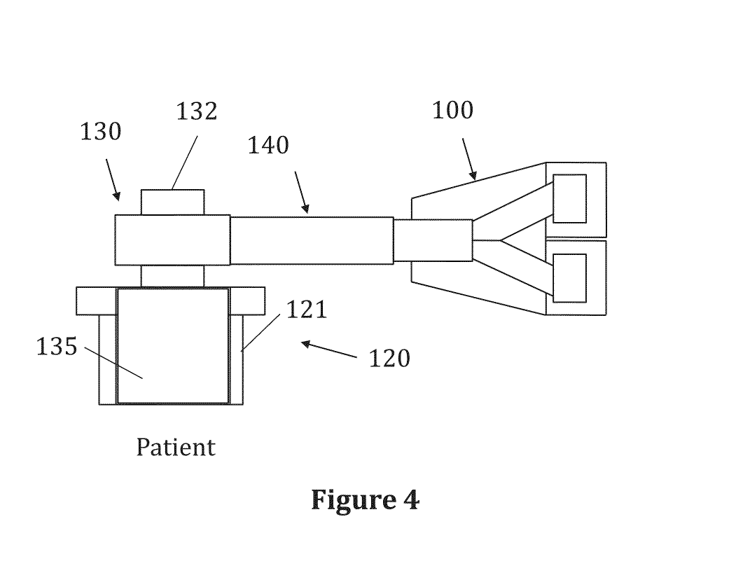

FIG. 4 is a schematic drawing of the high flow ventilation system with the gas mixing apparatus 100 and the bite block device 120 with the T-piece adapter 130 assembled together. In use, each jet mixing device of the gas mixing apparatus 100 is connected to a standard wall oxygen flow meter via a piece of flexible tubing. The outflow of the gas mixing apparatus 100 is connected in turn to the bite block device 120 via a piece of large diameter flexible tubing 140 that attaches to the inlet port of the T-piece adapter 130. The flow rate of oxygen is adjusted to the desired level and the bite block component 121 of the bite block device 120 is inserted into the patient's mouth and held in place by a strap. An endoscope can then be inserted into the endoscope port 132 and through the bite block device 120 into the patient. High flow, oxygen-enriched air will be delivered through the annular space between the endoscope and the central passage of the bite block device and into the patient's lungs.

The high flow ventilation system of FIG. 4 may also be configured to include an air reservoir, an air heater and a humidifier. Sensors, monitoring devices and anesthesia delivery equipment may also be used with the ventilation system.

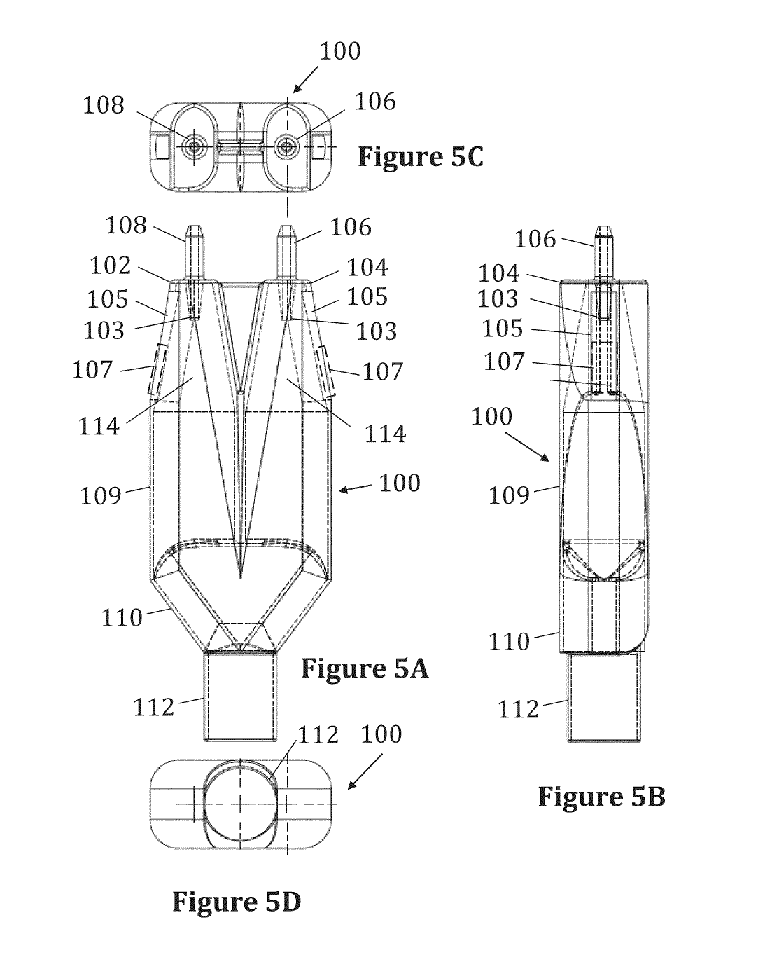

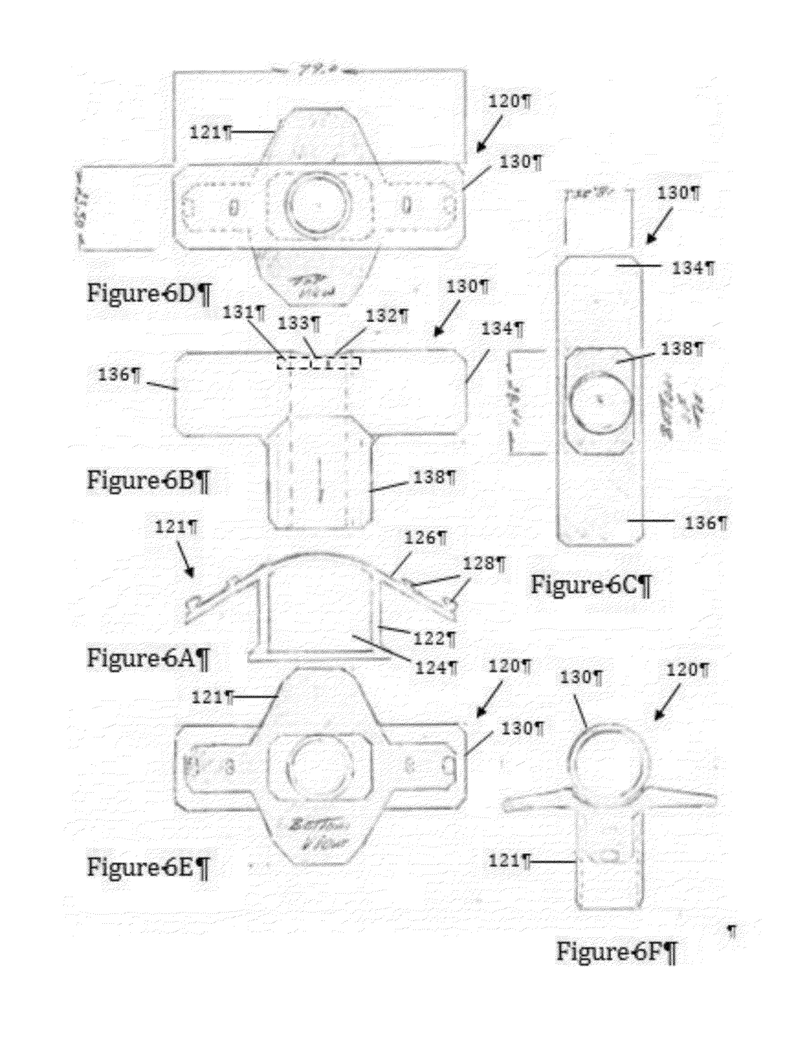

The apparatus of the present invention can be made in many different configurations that meet the functional requirements for a high flow ventilation system described above. By way of example, FIGS. 5A-5D illustrate a preferred embodiment of the gas mixing apparatus. FIG. 5A is a front view, FIG. 5B is a side view, FIG. 5C is a first end view and FIG. 5D is a second end view of the gas mixing apparatus 100. FIGS. 6A-6F are drawings of the components of a preferred embodiment of the bite block device 120.

The gas mixing apparatus 100 of FIGS. 5A-5D has a first jet mixing device 102 and a second jet mixing device 104 that are connected together by a Y-connector 110 having a single outflow port 112. Each jet mixing device has a tubing barb 106, 108 for connecting to a standard wall oxygen flow meter with a piece of flexible tubing. As described above, each jet mixing device has a small diameter jet orifice 103 in fluid connection with the tubing barb 106, 108 and positioned within a gas mixing chamber 114 enclosed by the tubular housing 109. One or more air entrainment ports 105 in the wall of the tubular housing 109 allow ambient air to be entrained into the housing and mixed with the oxygen. The size of the air entrainment ports 105 can be adjusted with a sliding, rotating or removable shutter 107 to regulate the ratio of oxygen to ambient air from 21% up to 100% oxygen. Each jet mixing device 102, 104 may have its own shutter 107 to adjust the size of its air entrainment ports 105 or a single shutter mechanism may be configured to adjust both jet mixing devices at once.

The bite block device 120 of FIGS. 6A-6F includes a bite block component 121 and a T-piece adapter 130. FIG. 6A shows a side profile view of the bite block component 121, which has a tubular body 122 with a central passage 124 and a flange 126 on one end. The flange 126 is curved to conform to the curvature of a patient's mouth and has a plurality of hook-shaped lugs 128 for fastening a strap to attach the bite block device 120 to the patient. Preferably, the bite block component 121 is molded of a flexible polymer or elastomer for the patient's comfort. FIG. 6B shows a front view and FIG. 6C shows a bottom view of a T-piece adapter 130. The T-piece adapter 130 has an endoscope port 132, an inlet port 134, an outlet port 136, and a connection port 138 that is configured to connect to the bite block component 121. The endoscope port 132 is furnished with a flexible diaphragm 131 with a central opening 133 to seal around the body of the endoscope when it is inserted, while allowing longitudinal movement of the endoscope. FIG. 6D is a top view, FIG. 6E is a bottom view and FIG. 6F is a side profile view of the bite block component 121 assembled together with the T-piece adapter 130.

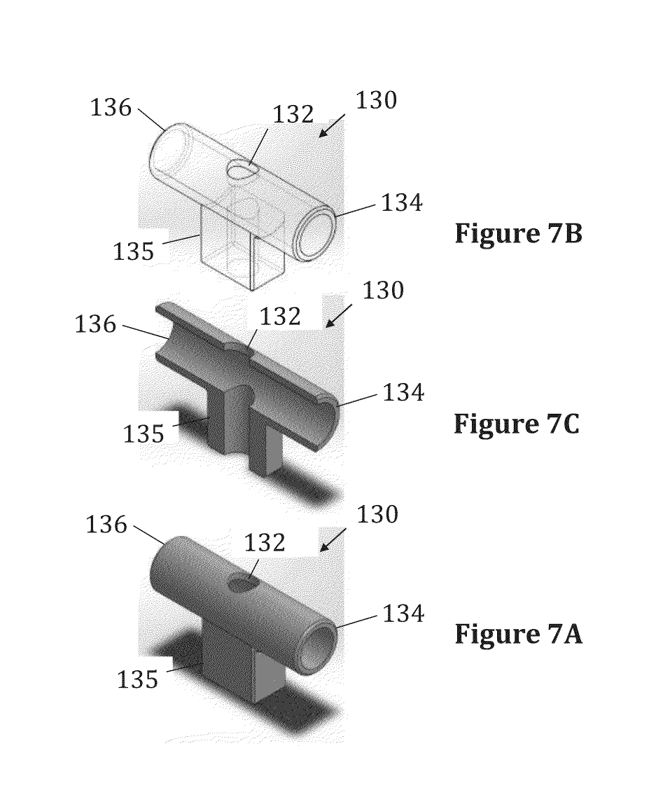

FIGS. 7A-7C show a version of the T-piece adapter 130 with the bite block insert 135 integrated directly into it. FIG. 7A is a perspective view of the T-piece adapter 130; FIG. 7B is a phantom view showing the interior structure of the T-piece adapter 130; and FIG. 7C is a cutaway view showing a cross section of the T-piece adapter 130.

The various components of the high flow ventilation system may be made by a variety of manufacturing processes, including machining, injection molding, rotational molding, casting, additive manufacturing, 3-D printing, etc, using medical grade polymers and elastomers or other suitable materials.

The gas mixing apparatus 100 and the bite block device 120 of the high flow ventilation system may be provided to hospitals and medical providers as separate components or they may be provided together in a kit or procedure tray, along with connection tubing and other necessary components. Preferably, the kit or procedure tray will be provided sterile and ready to use.

In alternative embodiments, the gas mixing apparatus 100 of the invention can also be used with a laryngeal mask airway, an endotracheal tube, an endoscopy mask or a standard ventilation mask. These components can also be provided in a kit or procedure tray for convenience of use.

What is claimed is:

1. A high flow ventilation system for delivering oxygen-enriched air to a patient, the system comprising:
a tubular housing with a wall enclosing a first gas mixing chamber and a second gas mixing chamber;
a first oxygen inlet for connection to a first source of pressurized oxygen;
a first jet orifice within the tubular housing, the first jet orifice being connected to the first oxygen inlet and directed into the first gas mixing chamber;
a first air entrainment port extending through the wall of the tubular housing and connecting to the first gas mixing chamber proximate to the first jet orifice;
a second oxygen inlet for connection to a second source of pressurized oxygen;
a second jet orifice within the tubular housing, the second jet orifice being connected to the second oxygen inlet and directed into the second gas mixing chamber;
a second air entrainment port extending through the wall of the tubular housing and connecting to the second gas mixing chamber proximate to the second jet orifice; and
an outflow port for oxygen-enriched air connected to the first gas mixing chamber and the second gas mixing chamber within the tubular housing.

2. The high flow ventilation system of claim 1, further comprising:
a first shutter for adjusting a size of the first air entrainment port; and
a second shutter for adjusting a size of the second air entrainment port.

3. The high flow ventilation system of claim 1, further comprising:
a bite block device configured to fit into the patient's mouth; and
a tube connecting the outflow port to the bite block device.

4. The high flow ventilation system of claim 3, wherein the bite block device has an endoscope port configured to allow passage of an endoscope body through the bite block device into the patient's mouth; and wherein the endoscope port of the bite block device has a flexible diaphragm with a central opening to seal around the body of the endoscope when it is inserted, while allowing longitudinal movement of the endoscope.

5. The high flow ventilation system of claim 4, wherein the bite block device provides an annular air flow passage around the endoscope for flow of oxygen-enriched air into the patient.

6. The high flow ventilation system of claim 4, wherein the bite block device includes a bite block and a T- piece adapter, a first arm of the T-piece adapter provides a connection for the flexible tube that connects the outflow port to the bite block device, a second arm of the T-piece adapter provides a connection for an air reservoir; and wherein the endoscope port is located on the T-piece adapter between the first arm and the second arm.

7. A method for ventilating a patient using the high flow ventilation system of claim 3, comprising:
connecting the first oxygen inlet to a first source of pressurized oxygen;
connecting the second oxygen inlet to a second source of pressurized oxygen;
allowing oxygen to flow through the first jet orifice into the first gas mixing chamber, thus entraining ambient air into the first gas mixing chamber;
allowing oxygen to flow through the second jet orifice into the second gas mixing chamber, thus entraining ambient air into the second gas mixing chamber;
combining an outflow of oxygen-enriched air from the first gas mixing chamber and the second gas mixing chamber into the outflow port; and
directing the oxygen-enriched air from the outflow port through the bite block device to the patient.

8. The method of claim 7, further comprising:
inserting an endoscope into the patient through an endoscope port in the bite block device.

9. The method of claim 8, further comprising:
providing the endoscope port of the bite block device with a flexible diaphragm with a central opening that forms a sliding seal around the body of the endoscope, while allowing longitudinal movement of the endoscope.

10. The method of claim 9, further comprising:
directing the oxygen-enriched air into the patient through an annular space between the endoscope and the bite block device.
